(19) United States
(12) Patent Application Publication
(10) Pub. No.: US 2011/0229430 A1
(43) Pub. Date: Sep. 22, 2011

(54) Cosmetic Compositions Containing Thiomers For Hair Color Retention

(76) Inventors: Geoffrey Hawkins, Yardley, PA (US); Isabelle R. Afriat, Maaleh Adumim (IL); Jean Harry Xavier, Holbrook, NY (US); Lavinia C. Popescu, Jackson Heights, NY (US)

(21) Appl. No.: 12/877,242

(22) Filed: Sep. 8, 2010

Related U.S. Application Data
(60) Provisional application No. 61/241,998, filed on Sep. 14, 2009.

(51) Int. Cl. A61K 8/899; A61K 8/84; A61K 8/81; A61K 8/73; A61K 8/90; A61K 8/87; A61K 8/85; A61Q 5/12; A61Q 5/00

(52) U.S. Cl. 424/70.12; 424/70.11; 424/70.15; 424/70.16; 424/70.13

(57) ABSTRACT

Cosmetic hair treatment compositions comprising thiolated polymers having in the range of from about 5% to about 50% reactive thiol groups are provided. Also provided is a method for extending the color retention of color-treated hair.

This application claims priority from U.S. 61/241,998, filed Sep. 14, 2009.

### BACKGROUND OF THE INVENTION

The present invention relates to a composition for treating human hair, in particular, post-hair dying treatment compositions which extend the color retention of color-treated hair. More specifically, the present invention relates to cosmetic compositions containing thiolated polymers (thiomers).

Hair color products are popular with consumers, but such products tend to damage the hair over time. Consumers are always looking for treatment products which will ensure true, long-lasting color (fade resistance) and hair damage resistance. It would also be desirable if such products could also repair the damage caused by hair coloring products, and also the damage inflicted on the hair by UV exposure, brushing, combing, heat from hair dryers, and the like. Consumers would also appreciate a product which would strengthen the hair.

The hair shaft is made up of two to three layers: the cuticle, the cortex, and sometimes the medulla. The cuticle is the outermost layer. It is made of flattened, overlapping, no longer living, cells. The cuticle protects the inside of the hair shaft from damage. The cortex underlies the cuticle. The cortex is made of long twisted proteins or keratins which give hair its strength and elasticity. When hair is stretched, these long proteins are straightened, and when hair is released, the proteins coil up again. The pigments which give natural hair its color are associated with these proteins and are protected by the outer cuticle. Combing, brushing, and environmental factors such as sun, air pollution, wind and water can damage the cuticle and cause the fibers of the cortex to fray, resulting in split ends. As the cortex cannot repair itself, to rid the hair of split ends, the damaged hair can only be cut off. The center of some hairs (especially coarse hair) includes a soft, spongy medulla.

Natural, healthy hair, that is, hair which has not been color-treated, or otherwise damaged, is further protected by a branched fatty acid, the “f-layer”, which is comprised of 18-methyl eicosanoic acid or 18-MEA. The fatty acid is covalently bonded to the surface of the hair cuticle and acts as a natural lubricating or conditioning system. This natural protection is hydrophobic (i.e., water-repellant), and contributes to the hair's smooth feel. Light reflected by smooth hair makes the hair appear glossy. The f-layer helps to protect the cuticle from damage, which may be caused by environmental factors, such as sun, wind and air pollution, or mechanical stresses, such as combing and brushing, or from heat, such as from blow-drying the hair. When the cuticle is damaged, its cells do not lie flat. Roughened cuticle surfaces make hair appear dull and unhealthy. Damaged hair also becomes tangled and is difficult to brush and to otherwise manage.

Perming, straightening, or coloring the hair can also damage the cuticle. As any hair color user recognizes, the coloring process changes the way their hair feels and behaves. The hair becomes rougher, drier and less shiny, and the color fades over time. The reason for these changes is that hair coloring alters the biology of the hair. The proteins, lipids and pigments in the hair are chemically altered. The combination of hydrogen peroxide, ammonia, and the high pH typical of conventional oxidative permanent colorants, applied to the hair surface bleaches the hair's natural pigment and introduces dye precursors and couplers which result in the formation of color. However, an undesirable side-effect of the hair-dying process is the removal of some of the protective f-layer which can lead to further oxidation of the hair surface and irreversible physiochemical changes in hair fibers. As the natural lubricating layer deteriorates, the hair becomes hydrophilic or water-loving instead of hydrophobic or water-repellent. Repeated hair coloring can lead to the disappearance of the f-layer altogether. The unprotected hair then becomes more susceptible to damage to the cuticle from mechanical stresses and heat, such as from combing, brushing and blow-drying the hair, and may feel dry, stiff, and coarse, and may be more difficult to detangle. As the hair becomes less water-repellent, due to the loss of the f-layer and damage to the cuticle, water penetrates through the hair cuticle and washes away the color causing the color of color-treated hair to fade. Hair looks duller and requires more frequent and/or more intense conditioning for manageability. Nevertheless, the effects of such conditioning treatments are temporary, as conditioners are washed away with each shampooing and must be reapplied.

Thiomer structure is different from the structures of polymers used in existing products because the thiomers consist of a polymer backbone, which may be cationic, non-ionic, anionic or silicone, which contains linear or branched thiol groups or substituents containing thiol groups.

### SUMMARY OF THE INVENTION

A need exists for cosmetic treatment compositions which will produce a water-resistant and friction-resistant film on the hair, restoring the hair's hydrophobicity, smoothness, manageability and brilliance. Such treatment compositions should be safe to use and should provide protection from damage, and extend the color retention of the hair after the hair dying process. These benefits should be retained long-term; that is, shampoo after shampoo. The inventors have discovered, surprisingly and unexpectedly, that compositions containing thiolated polymers will achieve these results better than any existing conditioning products.

The present invention provides a cosmetic hair treatment composition comprising at least one thiolated polymer having a degree of thiolation in the range of from greater than about 3% to about 50% (i.e., having from greater than about 3% to about 50% of reactive thiol groups) in a cosmetically or dermatologically acceptable vehicle. Preferably, the degree of thiolation is in the range of from about 5% to about 50%, more preferably in the range of from about 10% to about 30% reactive thiol groups.

The present invention further provides a method for treating the hair to repair or resurface damaged hair cuticles, thus restoring one or more of hydrophobicity, smoothness, manageability and brilliance of the hair, the method comprising applying to the hair in need of such treatment a cosmetic composition comprising at least one thiolated polymer having from greater than about 3% to about 50% of reactive thiol groups, in a cosmetically or dermatologically acceptable vehicle; and leaving the composition on the hair for a period of time sufficient to obtain the desired effect. Preferably, the at least one thiolated polymer has is in the range of from about 5% to about 50%, more preferably in the range of from about 10% to about 30%, reactive thiol groups.

The present invention also provides a method for extending the color retention of color-treated hair, which comprises applying to the hair in need of the extended color retention a composition comprising at least one thiolated polymer having from greater than about 3% to about 50% of reactive thiol groups, in a cosmetically or dermatologically acceptable vehicle; and leaving the composition on the hair for a period of time sufficient to obtain the desired effect. Preferably, the degree of thiolation is in the range of from about 5% to about 50%, more preferably in the range of from about 10% to about 30%.

### BRIEF DESCRIPTION OF THE DRAWINGS

FIG. 1 is a schematic representation illustrating the mechanism of action of the compositions and methods of the present invention.

FIG. 2 is a set of SEM images of hair surfaces.

FIG. 3 is a set of SEM images of hair surfaces.

FIG. 4 is bar graph illustrating the quantity of disulfide bonds on hair surfaces.

### DETAILED DESCRIPTION OF THE INVENTION

Conventional permanent hair color processes cause color to penetrate and become distributed throughout the hair cortex. However, the solubility of the dye and the degree of damage to the hair cuticle over time, including the loss of the f-layer, caused by hair coloring, facilitates the leaching of color from the cortex, especially as a result of shampooing, so that hair coloring must be reapplied about every four to six weeks, further damaging the cuticle.

Available conditioning products provide, at best, a temporary conditioning of the hair shaft, briefly restoring the friction-resistance of the hair, so that the hair temporarily looks and feels smoother and healthier. However, these products cannot arrest color loss. Color continues to escape from the cortex through the cuticle, resulting in the fading of the color of color-treated hair. Moreover, although available conditioning products are typically hydrophobic, they cannot restore the hydrophobicity of the hair shaft. This is because, being hydrophobic themselves, they cannot bind to the hydrophilic hair; that is, hair which has lost its hydrophobicity due to damage to the cuticle and/or loss of the f-layer. Color continues to wash out of the hair, resulting in hair color fading.

Thiolated polymers have been known for use as a promising tool in the field of mucoadhesive drug delivery and in tissue engineering applications. In view of the self-crosslinking properties of these polymers, and their affinity for thiol domains, the inventors investigated thiolated polymers for their possible efficacy in the color protection of chemically altered hair.

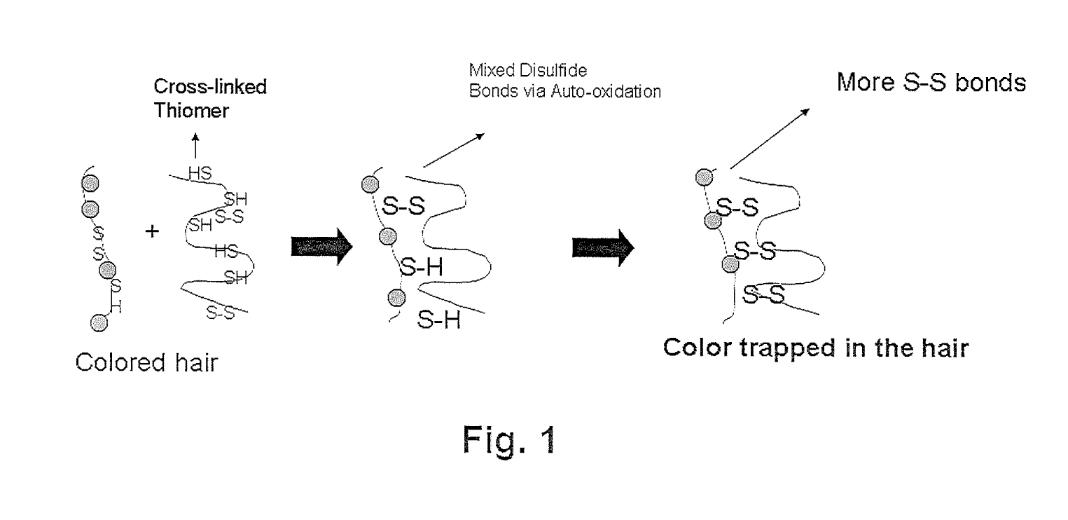

The major protein constituent of the hair cortex is keratin. Hair keratin is characterized by a content of cysteine residues of about 7.6%. Cysteine is a hydrophobic amino acid having a thiol (sulfhydryl or S—H) side chain. The thiol is susceptible to oxidation to give the disulfide derivative cystine which serves an important structural role (i.e., folding and stability) in many proteins. The amino acid cystine is present at about 5% in human hair keratin. As shown in FIG. 1, the thiolated polymer partially self-crosslinks through about 5-10% its thiol groups under atmospheric conditions via auto-oxidation to form disulfide bonds. When the composition of the present invention is applied to the hair, the remaining 90-95% thiol groups of the polymer react with the cysteine and cystine domains of the exposed keratin of the colored or damaged hair to form additional disulfide bonds. While not wishing to be bound by any particular theory, the inventors believe that it is the crosslinking through disulfide bonding of the thiolated polymer to the hair surface, and the resulting reduction in the number of available reactive thiol groups on the hair surface, which imparts the film, and thus the hair surface to which the polymer has been applied, with water-resistance, the crosslinking essentially resulting in a gel film formed on wet hair. Once the water evaporates from the hair, the thiolated polymer film is water-resistant; that is, not easily soluble in water, as the number of reactive thiol groups which could hydrogen bond with water molecules is minimized. Thus, the thiomer forms a hydrophobic protective layer that is covalently bonded to the hair and not readily solvated. As water less freely penetrates the film into the hair shaft, color is not easily washed away, even after several shampooings, for example, after 2-10 shampooings, and even after 30, 40 or even 50 shampooings.

The present invention aims to provide unique conditioning and color-locking breakthrough technology which not only repairs and resurfaces damaged hair cuticles, but which also seals in new hair color. The present invention achieves these objectives by providing cosmetic compositions for treating the hair which comprise at least one thiolated polymer. As used herein, “thiolated polymer” and “thiomer” mean a polymer or a copolymer having a degree of thiolation in the range of from greater than about 3% to about 50%; that is, the polymers will have in the range of from greater than about 3% to about 50% reactive thiol groups. The reactive thiol groups may be in the form of an end group or a pendant group of the polymer. Preferably, the degree of thiolation is in the range of from about 5% to about 50%, more preferably in the range of from about 10%, to about 30%.

Thiolated polymers useful in the compositions and methods of the present invention may be naturally hydrophobic or may be treated to render them hydrophobic. In one preferred embodiment of the present invention, the treatment composition comprises a blend of at least one thiolated polymer with at least one further polymer which is naturally hydrophobic or treated to render the polymer hydrophobic. In a further preferred embodiment of the present invention, the treatment composition comprises at least two thiolated polymers and, optionally, at least one further polymer or block copolymer which is hydrophobic or treated to render the polymer or copolymer hydrophobic.

The thiolated polymers suitable for use in the present invention may be anionic, cationic or non-ionic, or silicone, linear or branched homopolymers, hydrophobic block polymers or amphiphilic block copolymers, having in the range of from greater than about 3% to about 50% reactive thiol groups, preferably in the range of from about 5% to about 50%, and most preferably in the range of from about 10% to about 30%, and which are capable of forming a flexible film when applied to the hair, either alone or in a composition comprising the polymers. The reactive thiol groups of the polymers should have the ability to crosslink onto the hair cuticle, forming disulfide bonds with cysteine domains which are present in substantially greater amounts in damaged hair as compared with undamaged hair, to form a protective layer that is covalently bonded to the hair cuticle. The resulting film on hair should demonstrate strong water-, surfactant-, and wear-resistant characteristics to resist and preferably to prevent water/moisture from penetrating into the cortex and washing color away, thus fading the hair.

The thiolated polymers should be compatible with a variety of hair cosmetic formulations, including oil/water, silicon/water, water/oil, and water/silicone emulsions, or aqueous preparations. Suitable compositions may take the form of aqueous solutions, serums, gels, lotions, mousses, creams, and the like. The compositions of the present invention may take the form of an after-color treatment leave-on product, for example, a leave-on conditioning product. Also contemplated are shampoos, hair dying products, masques, hair styling products, a kit containing a hair dye product, a conditioning product, and/or a styling product, and the like.

Preferred polymers for use in the compositions of the present invention include, but are not limited to:

1. Linear or branched homopolymers having in the range of from greater than about 3% to about 50% reactive thiol groups, preferably, in the range of from about 5% to about 50%, more preferably in the range of from about 10% to about 30%, reactive thiol groups. Such homopolymers include, but are not limited to, polystyrene, polyester, polyfluoroester, polyethylene, polypropylene, polybutadiene, polyisoprene, polyurethane, polyimide, silicones, hydrophobically modified polyacrylates, for example, hydrophobically modified hyaluronic acid, hydrophobically modified cellulose, hydrophobically modified starch, hydrophobically modified polysaccharides, hydrophobically modified polyvinyl pyrrolidone, hydrophobically polyvinyl alcohol, and the like. An example of a modified hyaluronic acid is hyaluronic acid modified with cationic hydroxyethylcellulose, available as Biocare HA-24™, from Amerchol, or carboxymethyl hyaluronic acid, available as Glycosil™ from Glycosan Biosystems. In one preferred embodiment of the present invention, the homopolymer comprises a hydrophobic silicone backbone. In one embodiment of the present invention, the homopolymers are silicone homopolymers, more particularly, silicone mercapto polymers having the general structure:

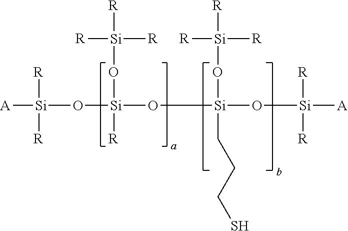

wherein A and each R are independently a C₁₋₃₀ straight or branched chain, saturated or unsaturated, alkyl, phenyl, aryl, trialkylsiloxy, and a and b are each about 20 to 50 and the ratio of a:b is in the range of from about 4:1-1:4. In one preferred embodiment of the present invention the ratio of a:b is about 1:1. Preferred is where ASiRR— is an alkyl siloxy, preferably, a methyl siloxy, endcap unit; in particular trimethylsiloxy and each R is a C₁₋₂₂ alkyl, phenyl, most preferably methyl or phenyl.

Non-limiting examples of silicone mercapto polymers for use in the compositions and methods of the present invention are Dimethicone/Mercapto propyl Methicone Copolymer, available as Gransil M-SH Fluid, and Dimethicone/Mercapto propyl Methicone Copolymer (and) Phenyl Trimethicone, available as Gransil PM-SH Fluid, both available from Grant Industries, Inc.

Dimethicone/Mercapto propyl Methicone Copolymer has the following general structure:

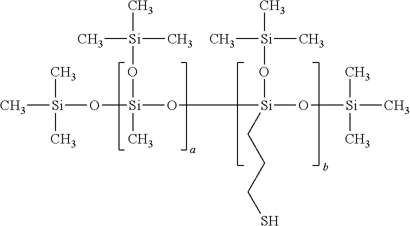

wherein each of a and b is a number in the range of from about 20 to about 50, and the ratio of a:b is preferably in the range of from about 4:1-1:4, such as about 1:1.

Dimethicone/Mercapto propyl Methicone Copolymer (and) Phenyl Trimethicone has the following general structure:

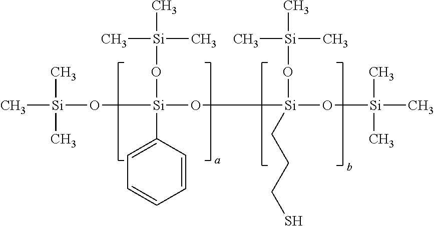

wherein each of a and b is a number in the range of from about 20 to about 50, and the ratio of a:b is preferably in the range of from about 4:1-1:4, such as about 1:1.

2. Linear or branched amphiphilic block copolymers comprising backbones of hydrophobic and hydrophilic blocks, including gradient copolymers, having in the range of from greater than about 3 wt. % to about 50 wt. % of reactive thiol groups, preferably, in the range of from about 5 wt. % to about 50 wt. %, more preferably in the range of from about 10 wt. % to about 30 wt. %, reactive thiol groups. The reactive thiol groups may be attached to the hydrophilic end or to hydrophilic blocks of the copolymer. The hydrophobic homopolymers suitable for use in the amphiphilic block copolymers include, but are not limited to, those listed hereinabove. Non-limiting examples of the hydrophilic blocks suitable for use in the block copolymers include polyvinyl pyrrolidone, hyaluronic acid, polyacrylates, polyvinyl chloride, polysaccharides, cellulose, and the like. Examples of such block copolymers include, but are not limited to C₁₂₋₂₂ methacrylates/acrylates copolymer, available as Allianz OPT™, from ISP; hydrophobic acrylates copolymer, available as Covacryl P12™ from Sensient Cosmetic Technologies; and polyquaternium-55 vinyl pyrrolidone/dimethylaminopropyl methacrylamide/methacryloylaminopropyl lauryl dimethyl ammonium chloride, available as Styleze W-17™ and Styleze W-20™ from ISP.

3. Polymeric blends may comprise, but are not limited to mixtures of one or more of any of the above-listed thiolated homopolymers or block copolymers blended with at least one further hydrophobic polymer. As examples of such hydrophobic polymers, use may be made of hydrophobically modified hyaluronic acid, hydrophobically modified cellulose, hydrophobically modified starch, hydrophobically modified polysaccharides, hydrophobically modified polyvinyl pyrrolidone, hydrophobically polyvinyl alcohol, and the like. In one embodiment of the invention, such a blend contains a linear thiolated hyaluronic acid polymer having about 25% reactive thiol groups blended with hydrophobically modified acrylates, silicones, or hydroxyethylcellulose block copolymers. Particularly preferred blends contain at least one mercapto silicone polymer, non-limiting examples of which include the following:
1. 50 wt. % Gransil M-SH fluid (Dimethicone/mercapto propyl methicone copolymer (11% reactive thiol pendant groups))
   20 wt. % DC 7-4405 (dimethicone silylate/isododecane film former)
   10 wt. % Covacryl P-12 (hydrophobically modified acrylate polymer)
   5 wt. % Fucogel II BPC (polysaccharide)
   5 wt. % Lipidure PMB pH 10 (Polyquaternium-51)
   10 wt. % vinyl caprolactam/EthylAminopropylethylamine copolymer
2. 50 wt. % Gransil PM-SH fluid (Dimethicone/Mercapto propyl Methicone Copolymer (and) Phenyl Trimethicone (11% reactive thiol pendant groups))
   20 wt. % DC 7-4405 (dimethicone silylate/isododecane film former)
   10 wt. % Covacryl P-12 (hydrophobically modified acrylate polymer)
   5 wt. % Fucogel II BPC (polysaccharide)
   5 wt. % Lipidure PMB pH 10 (Polyquaternium-51)
   10 wt. % vinyl caprolactam/EthylAminopropylethylamine copolymer
3. 50 wt. % Gransil M-SH fluid (Dimethicone/Mercapto propyl Methicone copolymer (11% reactive thiol pendant groups))
   20 wt. % DC 7-4405 (dimethicone silylate/isododecane film former)
   10 wt. % Covacryl P-12 (hydrophobically modified acrylate polymer)
   2 wt. % Fucogel II BPC (polysaccharide)
   2.5 wt. % Aquaflex XL-30 (polyimide-1)
   5 wt. % Lipidure PMB pH 10 (Polyquaternium-51)
   10 wt. % vinyl caprolactam/EthylAminopropylethylamine copolymer)
4. 40 wt. % Gransil PM-SH fluid (Dimethicone/Mercapto propyl Methicone Copolymer (and) Phenyl Trimethicone (11% reactive thiol pendant groups))
   20 wt. % DC 7-4405 (dimethicone silylate/isododecane film former)
   19.5 wt. % Aquaflex XL-30 (polyimide-1)
   5 wt. % vinyl caprolactam/EthylAminopropylethylamine copolymer
5. 40 wt. % Gransil PM-SH fluid (Dimethicone/Mercapto propyl Methicone Copolymer (and) Phenyl Trimethicone (11% reactive thiol pendant groups))
   20 wt. % DC 7-4405 (dimethicone silylate/isododecane film former)
   19.5 wt. % Aquastyle 3000 (polyvinylamide-1)
   5 wt. % vinyl caprolactam/ethylaminopropylethylamine copolymer)
6. 30 wt. % Gransil PM-SH fluid (Dimethicone/Mercapto propyl Methicone Copolymer (and) Phenyl Trimethicone (11% reactive thiol pendant groups))
   20 wt. % DC 7-4405 (dimethicone silylate/isododecane film former)
   19.5 wt. % Aquaflex XL-30 (polyimide-1)
   5 wt. % vinyl caprolactam/EthylAminopropylethylamine copolymer
7. 30 wt. % Gransil PM-SH fluid (Dimethicone/Mercapto propyl Methicone Copolymer (and) Phenyl Trimethicone (11% reactive thiol pendant groups))
   20 wt. % DC 7-4405 (dimethicone silylate/isododecane film former)
   19.5 wt. % Aquastyle 3000 (polyvinylamide-1)
   5 wt. % vinyl caprolactam/EthylAminopropylethylamine copolymer
8. 25 wt. % Gransil M-SH fluid (Dimethicone/mercapto propyl Methicone copolymer (11% reactive thiol pendant groups))
   25 wt. % Gransil PM-SH fluid (Dimethicone/Mercapto propyl Methicone Copolymer (and) Phenyl Trimethicone (11% reactive thiol pendant groups))
   50 wt. % DC7-4405 (dimethicone silylate/isododecane film former)

The at least one thiolated polymer or polymer blend containing the at least one thiolated polymer may be present in the compositions of the invention in amounts in the range of from about 5 wt. % to about 50 wt. %, and may be, for example, about 5 wt. %, about 20 wt. %, about 30 wt. %, about 40 wt. %, about 50 wt. %, and may include any amount in between, by total weight of the composition. The total weight of the at least one thiolated polymer in the polymer blend containing the at least one thiolated polymer may be in the range of from about 30 wt. % to about 80 wt. %, including any amount in between, more preferably in the range of from about 30 wt. % to about 50 wt. %, by total weight of the polymer blend.

In a particularly preferred embodiment, compositions of the present invention will include one or more styling/fixative aids to enhance the performance of the thiolated polymers by facilitating the adherence of the thiolated polymers on the hair and by contributing a desired balance of flexibility/rigidity to the product. Styling/fixative aids suitable for use in the compositions of the present invention include, but are not limited to, film forming agents including, water-soluble acrylates copolymers, such as ethyl acrylate/methylmethacrylate/methacrylic acid copolymers, for example Covacryl A15® and Covacryl E14®, available from Sensient Cosmetic Technologies, vinylcaprolactam/ethyl aminopropylethylamine copolymer (available as Styleze™ CC-10, Copolymer 845, Copolymer 937, or Copolymer 958, vinylcaprolactam/Vinyl Pyrrolidone/Dimethyl aminopropylethylamine copolymer, available as Gaffix VC-713, Advantage S, or Advantage Plus, Acrylates/C12-22 Alkylmethacrylate Copolymer, available as Allianz OPT; silicone fluids, such as Dow Corning DC7-4405; polyimides such as polyimide-1, available as Aquaflex® XL-30 from ISP, Imidized Isobutylene/Maleic Anhydride, available as Aquaflex FX-64, Polyquaternium-55, available as Styleze W-17, Polyquaternium-11, available as Gafquat 734 or Gafquat 755N, Polyquaternium-16, available as Luviquat Style, Polyquaternium-10, available as UCARE JR-400, and the like. Such styling/fixative aids may be present in the compositions of the present invention in amounts in the range of from about 0.1 wt. % to about 50 wt. %, such as from about 2 wt. % to about 40 wt. %, by total weight of the composition.

Compositions of the present invention may further include ingredients such as moisturizers and/or conditioning agents such as pantethine, panthenyl ethyl ether, biopolysaccharide gum-1, available as Fucogel®, Polyquaternium-51, available as Lipidure®, glycerine, Polyquaternium-7, water-dispersible polysaccharides such as glycosaminoglycans, glucoaminoglycans, glycoaminoglycans, esters, such as tricaprylyl citrate, and the like. Such ingredients may be present in the compositions of the present invention in amounts in the range of from about 0.1 wt. % to about 25 wt. %, more preferably from about 0.5 wt. % to about 10 wt. %, by total weight of the composition.

Other compounds which may be found in the compositions of the present invention include, but are not limited to: buffers and salts to adjust the pH of the solution; preservatives and anti-microbial agents, such as Botanistat PF-64, available from D-D Chemco, Inc.; antioxidants, such as vitamin C, DNA repair extracts encapsulated in liposomes, such as Roxisomes® (Arabidposis Exact/lecithin/water/phenoxyethanol), Ultrasomes® (Micrococcus lysate); vitamins such as vitamin A or vitamin E; nutrients both essential and non-essential, such as amino acids and minerals; and compounds that protect against environmental insult and toxins such as ultraviolet light and pollution. It may also be desirable to include one or more humectants in the composition. If present, such humectants may range from about 0.001 to 25%, preferably from about 0.005 to 20%, more preferably from about 0.1 to 15%, by total weight of the composition. Examples of suitable humectants include glycols, sugars, and the like. Suitable glycols are in monomeric or polymeric form and include polyethylene and polypropylene glycols such as PEG 4-200, which are polyethylene glycols having from 4 to 200 repeating ethylene oxide units; as well as C₁₋₆ alkylene glycols such as propylene glycol, butylene glycol, pentylene glycol, and the like. Suitable sugars, some of which are also polyhydric alcohols, are also suitable humectants. Examples of such sugars include glucose, fructose, honey, hydrogenated honey, inositol, maltose, mannitol, maltitol, sorbitol, sucrose, xylitol, xylose, and so on. Also suitable is urea. The humectants used in the composition of the invention may be C₁₋₆, preferably C₂₋₄ alkylene glycols, such as butylene glycol. A preferred humectant used in the compositions of the invention is glycerin.

Compositions of the present invention may preferably also comprise one or more anti-static agents including behentrimonium methyl sulfate, stearalkonium chloride, polyquaternium-10, and so forth. Conditioning agents useful in the compositions of the present invention include one or more of cetearyl alcohol/behentrimonium chloride, tricaprylyl citrate, sodium gluconate, hydroxy propyl starch phosphate, and the like. These other compounds will be present in the range of from about 0.0001 to about 40%, by total weight of the composition.

Compositions of the present invention may include one or more botanical ingredients or actives, including oils and extracts, such as, but not limited to, Helianthus annuus (sunflower) seed oil, Macadamia terifolia seed oil, Foeniculum vulgare (fennel) seed extract, Aspalathus linearis (rooibos) leaf extract, Simmondsia chinensis (jojoba) seed oil, Ricinus communis (castor) seed oil, and the like. Such materials may be present in the compositions of the invention in amounts in the range of from about 0.0001 to about 40%, by total weight of the composition.

If emulsions, the compositions of the present invention may be water-in-oil, oil-in-water, silicone-in-water, or water-in-silicone, comprising from about 0.1 to 95%, preferably from about 0.5 to about 90%, and more preferably from about 1 to 85% water, by total weight of the composition. If in the form of aqueous solutions, suspensions or gels, the composition may contain from about 10 to about 99% water, by total weight of the composition, with the remaining ingredients being one or more actives.

In the event the composition of the invention is an emulsion, the composition will comprise an oil phase. Oily ingredients are desirable for the skin moisturizing and protective properties. Suitable oils include silicones, esters, vegetable oils, including but not limited to those set forth herein. The oils may be volatile or nonvolatile, and are preferably in the form of a pourable liquid at room temperature. The term “volatile” means that the oil has a measurable vapor pressure or a vapor pressure of at least about 2 mm of mercury at 20° C. The term “nonvolatile” means that the oil has a vapor pressure of less than about 2 mm of mercury at 20° C.

Cyclic silicones are one type of volatile silicone that may be used in the composition. Such silicones have the general formula:

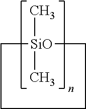

where n=3-6, preferably 4, 5, or 6.

Also suitable are linear volatile silicones, for example, those having the general formula:

(CH₃)₃—Si—O—[Si—(CH₃)₂—O]ₙ—Si(CH₃)₃

where n=0, 1, 2, 3, 4, or 5, preferably 0, 1, 2, 3, or 4.

Cyclic and linear volatile silicones are available from various commercial sources including Dow Corning Corporation and General Electric. The Dow Corning linear volatile silicones are sold under the tradenames Dow Corning 244, 245, 344, 345 and 200 fluids. These fluids include hexamethyldisiloxane (viscosity 0.65 centistokes (abbreviated cst)), octamethyltrisiloxane (1.0 cst), decamethyltetrasiloxane (1.5 cst), dodecamethylpentasiloxane (2 cst) and mixtures thereof, with all viscosity measurements being at 25° C. A preferred cyclic volatile silicone is cyclopentasiloxane, available from Dow Corning as DC 345 Fluid.

Suitable branched volatile silicones include alkyl trimethicones such as methyl trimethicone, a branched volatile silicone having the general formula:

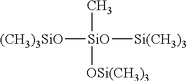

Methyl trimethicone may be purchased from Shin-Etsu Silicones under the tradename TMF-1.5, having a viscosity of 1.5 cst at 25° C.

Nonvolatile silicone oils, both water soluble and water insoluble, are also suitable for use in the composition. Such silicones preferably have a viscosity ranging from about greater than 5 to 800,000 cst, preferably 20 to 200,000 cst at 25° C. Suitable water insoluble silicones include amine functional silicones such as amodimethicone.

For example, such nonvolatile silicones may have the following general formula:

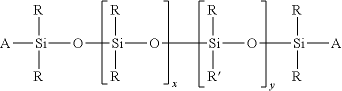

wherein R and R′ are each independently C₁₋₃₀ straight or branched chain, saturated or unsaturated alkyl, phenyl or aryl, trialkylsiloxy, and x and y are each independently 1-1,000,000; with the proviso that there is at least one of either x or y, and A is alkyl siloxy endcap unit. Preferred is where A is a methyl siloxy endcap unit; in particular trimethylsiloxy, and R and R′ are each independently a C₁₋₃₀ straight or branched chain alkyl, phenyl, or trimethylsiloxy, more preferably a C₁₋₂₂ alkyl, phenyl, or trimethylsiloxy, most preferably methyl, phenyl, or trimethylsiloxy, and resulting silicone is dimethicone, phenyl dimethicone, diphenyl dimethicone, phenyl trimethicone, or trimethylsiloxyphenyl dimethicone. Other examples include alkyl dimethicones such as cetyl dimethicone, and the like wherein at least one R is a fatty alkyl (C₁₂, C₁₄, C₁₆, C₁₈, C₂₀, or C₂₂), and the other R is methyl, and A is a trimethylsiloxy endcap unit, provided such alkyl dimethicone is a pourable liquid at room temperature. Dimethicone can be purchased from Dow Corning Corporation as DC 200/100 cs fluid. Preferred is a film forming polymer obtained by polycondensation of dimethiconol and MQ silicate resin in a solvent, such as isododecane, available from DC under the trade name DC7-4405 low Tack®.

A variety of nonvolatile oils are also suitable for use in the compositions of the invention. The nonvolatile oils generally have a viscosity of greater than about 5 to 10 centistokes at 25° C., and may range in viscosity up to about 1,000,000 centipoise at 25° C. Examples of nonvolatile oils include, but are not limited to esters and hydrocarbon oils. Suitable esters are mono-, di-, and triesters. The composition may comprise one or more esters selected from the group, or mixtures thereof.

Monoesters are defined as esters formed by the reaction of a monocarboxylic acid having the formula R—COOH, wherein R is a straight or branched chain saturated or unsaturated alkyl having 2 to 45 carbon atoms, or phenyl; and an alcohol having the formula R—OH wherein R is a straight or branched chain saturated or unsaturated alkyl having 2-30 carbon atoms, or phenyl. Both the alcohol and the acid may be substituted with one or more hydroxyl groups. Either one or both of the acid or alcohol may be a “fatty” acid or alcohol, and may have from about 6 to 30 carbon atoms, more preferably 12, 14, 16, 18, or 22 carbon atoms in straight or branched chain, saturated or unsaturated form. Examples of monoester oils that may be used in the compositions of the invention include hexyl laurate, butyl isostearate, hexadecyl isostearate, cetyl palmitate, isostearyl neopentanoate, stearyl heptanoate, isostearyl isononanoate, stearyl lactate, stearyl octanoate, stearyl stearate, isononyl isononanoate, and so on.

Suitable diesters are the reaction product of a dicarboxylic acid and an aliphatic or aromatic alcohol or an aliphatic or aromatic alcohol having at least two substituted hydroxyl groups and a monocarboxylic acid. The dicarboxylic acid may contain from 2 to 30 carbon atoms, and may be in the straight or branched chain, saturated or unsaturated form. The dicarboxylic acid may be substituted with one or more hydroxyl groups. The aliphatic or aromatic alcohol may also contain 2 to 30 carbon atoms, and may be in the straight or branched chain, saturated, or unsaturated form. Preferably, one or more of the acid or alcohol is a fatty acid or alcohol, i.e. contains 12-22 carbon atoms. The dicarboxylic acid may also be an alpha hydroxy acid. The ester may be in the dimer or trimer form. Examples of diester oils that may be used in the compositions of the invention include diisotearyl malate, neopentyl glycol dioctanoate, dibutyl sebacate, dicetearyl dimer dilinoleate, dicetyl adipate, diisocetyl adipate, diisononyl adipate, diisostearyl dimer dilinoleate, diisostearyl fumarate, diisostearyl malate, dioctyl malate, and so on.

Suitable triesters comprise the reaction product of a tricarboxylic acid and an aliphatic or aromatic alcohol or alternatively the reaction product of an aliphatic or aromatic alcohol having three or more substituted hydroxyl groups with a monocarboxylic acid. As with the mono- and diesters mentioned above, the acid and alcohol contain 2 to 30 carbon atoms, and may be saturated or unsaturated, straight or branched chain, and may be substituted with one or more hydroxyl groups. Preferably, one or more of the acid or alcohol is a fatty acid or alcohol containing 12 to 22 carbon atoms. Examples of triesters include esters of arachidonic, citric, or behenic acids, such as triarachidin, tributyl citrate, triisostearyl citrate, tri C₁₂₋₁₃ alkyl citrate, tricaprylin, tricaprylyl citrate, tridecyl behenate, trioctyldodecyl citrate, tridecyl behenate; or tridecyl cocoate, tridecyl isononanoate, and so on.

Esters suitable for use in the composition are further described in the C.T.F.A. Cosmetic Ingredient Dictionary and Handbook, Eleventh Edition, 2006, under the classification of “Esters”, the text of which is hereby incorporated by reference in its entirety.

It may be desirable to incorporate one or more nonvolatile hydrocarbon oils into the composition. Suitable nonvolatile hydrocarbon oils include paraffinic hydrocarbons and olefins, preferably those having greater than about 20 carbon atoms. Examples of such hydrocarbon oils include C₂₄₋₂₈ olefins, C₃₀₋₄₅ olefins, C₂₀₋₄₀ isoparaffins, hydrogenated polyisobutene, polyisobutene, polydecene, hydrogenated polydecene, mineral oil, pentahydrosqualene, squalene, squalane, and mixtures thereof. In one preferred embodiment such hydrocarbons have a molecular weight ranging from about 300 to 1000 Daltons.

Surface active agents which may be used in the compositions of the invention include silicone surfactants and organic nonionic surfactants. If used, surface active agents are present in the range of from about 0.1 to about 80%, preferably in the range of from about 1 to 50%, and more preferably in the range of from about 5 to about 40%, based on the total weight of the composition.

Suitable silicone surfactants include polyorganosiloxane polymers that have amphiphilic properties, for example contain hydrophilic radicals and lipophilic radicals. These silicone surfactants may be liquids or solids at room temperature.

One type of silicone surfactant that may be used is generally referred to as dimethicone copolyol or alkyl dimethicone copolyol. This surfactant is either a water-in-oil or oil-in-water surfactant having a Hydrophile/Lipophile Balance (HLB) ranging from about 2 to 18. Preferably the silicone surfactant is a nonionic surfactant having an HLB ranging from about 2 to 12, preferably about 2 to 10, most preferably about 4 to 6. The term “hydrophilic radical” means a radical that, when substituted onto the organosiloxane polymer backbone, confers hydrophilic properties to the substituted portion of the polymer. Examples of radicals that will confer hydrophilicity are hydroxy-polyethyleneoxy, hydroxyl, carboxylates, and mixtures thereof. The term “lipophilic radical” means an organic radical that, when substituted onto the organosiloxane polymer backbone, confers lipophilic properties to the substituted portion of the polymer. Examples of organic radicals that will confer lipophilicity are C₁₋₄₀ straight or branched chain alkyl, fluoro, aryl, aryloxy, C₁₋₄₀ hydrocarbyl acyl, hydroxy-polypropyleneoxy, or mixtures thereof.

One type of suitable silicone surfactant has the general formula:

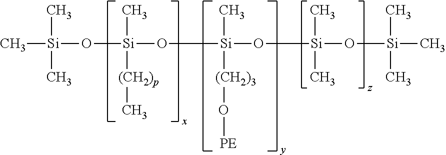

wherein p is 0-40 (the range including all numbers between and subranges such as 2, 3, 4, 13, 14, 15, 16, 17, 18, etc.), and PE is (—C₂H₄O)ₐ—(—C₃H₆O)_{b}—H wherein a is 0 to 25, b is 0-25 with the proviso that both a and b cannot be 0 simultaneously, x and y are each independently ranging from 0 to 1 million with the proviso that they both cannot be 0 simultaneously. In one preferred embodiment, x, y, z, a, and b are such that the molecular weight of the polymer ranges from about 5,000 to about 500,000, more preferably from about 10,000 to 100,000, and is most preferably approximately about 50,000 and the polymer is generically referred to as dimethicone copolyol. One type of silicone surfactant is wherein p is such that the long chain alkyl is cetyl or lauryl, and the surfactant is called, generically, cetyl dimethicone copolyol or lauryl dimethicone copolyol respectively.

In some cases the number of repeating ethylene oxide or propylene oxide units in the polymer are also specified, such as a dimethicone copolyol that is also referred to as PEG-15/PPG-10 dimethicone, which refers to a dimethicone having substituents containing 15 ethylene glycol units and 10 propylene glycol units on the siloxane backbone. It is also possible for one or more of the methyl groups in the above general structure to be substituted with a longer chain alkyl (e.g. ethyl, propyl, butyl, etc.) or an ether such as methyl ether, ethyl ether, propyl ether, butyl ether, and the like.

Examples of silicone surfactants are those sold by Dow Corning under the tradename Dow Corning 3225C Formulation Aid having the CTFA name cyclotetrasiloxane (and) cyclopentasiloxane (and) PEG/PPG-18 dimethicone; or 5225C Formulation Aid, having the CTFA name cyclopentasiloxane (and) PEG/PPG-18/18 dimethicone; or Dow Corning 190 Surfactant having the CTFA name PEG/PPG-18/18 dimethicone; or Dow Corning 193 Fluid, Dow Corning 5200 having the CTFA name lauryl PEG/PPG-18/18 methicone; or Abil EM 90 having the CTFA name cetyl PEG/PPG-14/14 dimethicone sold by Goldschmidt; or Abil EM 97 having the CTFA name bis-cetyl PEG/PPG-14/14 dimethicone sold by Goldschmidt; or Abil WE 09 having the CTFA name cetyl PEG/PPG-10/1 dimethicone in a mixture also containing polyglyceryl-4 isostearate and hexyl laurate; or KF-6011 sold by Shin-Etsu Silicones having the CTFA name PEG-11 methyl ether dimethicone; KF-6012 sold by Shin-Etsu Silicones having the CTFA name PEG/PPG-20/22 butyl ether dimethicone; or KF-6013 sold by Shin-Etsu Silicones having the CTFA name PEG-9 dimethicone; or KF-6015 sold by Shin-Etsu Silicones having the CTFA name PEG-3 dimethicone; or KF-6016 sold by Shin-Etsu Silicones having the CTFA name PEG-9 methyl ether dimethicone; or KF-6017 sold by Shin-Etsu Silicones having the CTFA name PEG-10 dimethicone; or KF-6038 sold by Shin-Etsu Silicones having the CTFA name lauryl PEG-9 polydimethylsiloxyethyl dimethicone.

Also suitable are various types of crosslinked silicone surfactants that are often referred to as emulsifying elastomers. They are typically prepared as set forth above with respect to the section “silicone elastomers” except that the silicone elastomers will contain at least one hydrophilic moiety such as polyoxyalkylenated groups. Typically these polyoxyalkylenated silicone elastomers are crosslinked organopolysiloxanes that may be obtained by a crosslinking addition reaction of diorganopolysiloxane comprising at least one hydrogen bonded to silicon and of a polyoxyalkylene comprising at least two ethylenically unsaturated groups. In at least one embodiment, the polyoxyalkylenated crosslinked organo-polysiloxanes are obtained by a crosslinking addition reaction of a diorganopolysiloxane comprising at least two hydrogens each bonded to a silicon, and a polyoxyalkylene comprising at least two ethylenically unsaturated groups, optionally in the presence of a platinum catalyst, as described, for example, in U.S. Pat. No. 5,236,986 and U.S. Pat. No. 5,412,004, U.S. Pat. No. 5,837,793 and U.S. Pat. No. 5,811,487, the contents of which are incorporated by reference.

Polyoxyalkylenated silicone elastomers that may be used in at least one embodiment of the invention include those sold by Shin-Etsu Silicones under the names KSG-21, KSG-20, KSG-30, KSG-31, KSG-32, KSG-33; KSG-210 which is dimethicone/PEG-10/15 crosspolymer dispersed in dimethicone; KSG-310 which is PEG-15 lauryl dimethicone crosspolymer; KSG-320 which is PEG-15 lauryl dimethicone crosspolymer dispersed in isododecane; KSG-330 (the former dispersed in triethylhexanoin), KSG-340 which is a mixture of PEG-10 lauryl dimethicone crosspolymer and PEG-15 lauryl dimethicone crosspolymer.

Also suitable are polyglycerolated silicone elastomers like those disclosed in PCT/WO 2004/024798, which is hereby incorporated by reference in its entirety. Such elastomers include Shin-Etsu's KSG series, such as KSG-710 which is dimethicone/polyglycerin-3 crosspolymer dispersed in dimethicone; or lauryl dimethicone/polyglycerin-3 crosspolymer dispersed in a variety of solvent such as isododecane, dimethicone, triethylhexanoin, sold under the Shin-Etsu tradenames KSG-810, KSG-820, KSG-830, or KSG-840. Also suitable are silicones sold by Dow Corning under the tradenames 9010 and DC9011. One preferred crosslinked silicone elastomer emulsifier is dimethicone/PEG-10/15 crosspolymer, which provides excellent aesthetics due to its elastomeric backbone, but also surfactancy properties.

The composition may comprise one or more nonionic organic surfactants. Suitable nonionic surfactants include alkoxylated alcohols, or ethers, formed by the reaction of an alcohol with an alkylene oxide, usually ethylene or propylene oxide. Preferably the alcohol is either a fatty alcohol having 6 to 30 carbon atoms. Examples of such ingredients include Steareth 2-100, which is formed by the reaction of stearyl alcohol and ethylene oxide and the number of ethylene oxide units ranges from 2 to 100; Beheneth 5-30 which is formed by the reaction of behenyl alcohol and ethylene oxide where the number of repeating ethylene oxide units is 5 to 30; Ceteareth 2-100, formed by the reaction of a mixture of cetyl and stearyl alcohol with ethylene oxide, where the number of repeating ethylene oxide units in the molecule is 2 to 100; Ceteth 1-45 which is formed by the reaction of cetyl alcohol and ethylene oxide, and the number of repeating ethylene oxide units is 1 to 45, and so on.

Other alkoxylated alcohols are formed by the reaction of fatty acids and mono-, di- or polyhydric alcohols with an alkylene oxide. For example, the reaction products of C₆₋₃₀ fatty carboxylic acids and polyhydric alcohols which are monosaccharides such as glucose, galactose, methyl glucose, and the like, with an alkoxylated alcohol. Examples include polymeric alkylene glycols reacted with glyceryl fatty acid esters such as PEG glyceryl oleates, PEG glyceryl stearate; or PEG polyhydroxyalkanotes such as PEG dipolyhydroxystearate wherein the number of repeating ethylene glycol units ranges from 3 to 1000.

Also suitable as nonionic surfactants are those formed by the reaction of a carboxylic acid with an alkylene oxide or with a polymeric ether. The resulting products have the general formula:

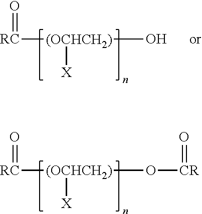

where RCO is the carboxylic ester radical, X is hydrogen or lower alkyl, and n is the number of polymerized alkoxy groups. In the case of the diesters, the two RCO-groups do not need to be identical. Preferably, R is a C6-30 straight or branched chain, saturated or unsaturated alkyl, and n is from 1-100.

Monomeric, homopolymeric, or block copolymeric ethers are also suitable as nonionic surfactants. Typically, such ethers are formed by the polymerization of monomeric alkylene oxides, generally ethylene or propylene oxide. Such polymeric ethers have the following general formula:

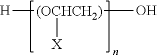

wherein X is H or lower alkyl and n is the number of repeating monomer units, and ranges from 1 to 500.

Other suitable nonionic surfactants include alkoxylated sorbitan and alkoxylated sorbitan derivatives. For example, alkoxylation, in particular ethoxylation of sorbitan provides polyalkoxylated sorbitan derivatives. Esterification of polyalkoxylated sorbitan provides sorbitan esters such as the polysorbates. For example, the polyalkyoxylated sorbitan can be esterified with C₆₋₃₀, preferably C₁₂₋₂₂ fatty acids. Examples of such ingredients include Polysorbates 20-85, more specifically Polysorbate 80, sorbitan oleate, sorbitan sesquioleate, sorbitan palmitate, sorbitan sesquiisostearate, sorbitan stearate, and so on.

Without intending to restrict in any way the scope of the invention, the following examples are presented to illustrate the invention's aspects and its use.

### Example 1

### Leave-on Composition

| Material | Weight Percent |
|---|---|
| *Thiolated hyaluronic acid | 1.5 |
| Citric acid (1% solution in water) | to adjust pH to 3.5 |
| Water | qs to 100 |

*25% reactive thiol groups; molecular weight, 150,000 Daltons

### Example 2

### Hydrophobicity/Hydrophilicity Analysis

The wetting property (hydrophobicity/hydrophilicity) of hair fiber was determined by measuring the water contact angle to the hair fiber surface. Non-colored virgin grey hair was used as the control.

Procedure for Measuring Contact Angle:

1. Hair swatch was colored as follows:
15-20 g swatch of grey hair was colored using commercial hair color-1 according to package directions.
The hair color was allowed to develop on the hair swatch for 25 minutes at room temperature.
The hair swatch was washed in the sink with tap running water until the water became completely clear.
The hair swatch was air dried for 30 minutes.
A single hair of every sample was mounted on a fiber holder and placed on sample stage of OCA 20 device, using a 50 μl micro syringe filled with filtered distilled water and glass dosing needle. Droplets of about 10 nanoliters of water were dispensed on the hair surface.
The placement procedure of the drop onto the sample surface was recorded using an automated video recording function. A total of 60 seconds of images were captured using a recording rate of 5 images/sample. The contact angles were automatically calculated for each image using manual fitting.
   2. Colored hair swatch was conditioned as follows:
2.5-3 g (20-25 cm length and 1 cm width) of colored hair swatch (from step 1, above) was damp dried with a paper towel.
1-1.2 g of Test Material 9 (see Table 2 below) was applied thoroughly by hand through the hair swatch and allowed to set or form a film for 1-2 minutes.
The swatch was washed in the sink with tap running water until the water became completely clear.
The swatch was damp dried with a paper towel, combed and blown dry for 10 minutes.
   Averages of four initial contact angle measurements for each sample are reported in Table 1.

TABLE 1

| | Water Con- | |
|---|---|---|
| | tact Angle | Hydrophobicity/ |
| Sample | (degrees) | Hydrophilicity |
| Virgin Grey Hair | 103.7 ± 2.2 | Hydrophobic |
| Virgin Grey Hair + commercial hair | 65 ± 1.2 | Hydrophilic |
| color-1¹ | | |
| Virgin Grey Hair + commercial hair | 84.5 ± 1.5 | Semi- |
| color-1¹ + commercial conditioner-1² | | Hydrophobic |
| Virgin Grey + commercial hair | 115 ± 3.6 | Hydrophobic |
| color-1¹ + Test Material 9 | | |
| Virgin Grey Hair + commercial hair | 127 ± 2.1 | Hydrophobic |
| color-1¹ + 4% Test Material 9/96% | | |
| commercial conditioner-1² | | |
| Virgin Grey Hair + commercial hair | 120 ± 3.4 | Hydrophobic |
| color-1¹ + 4% Test Material 9/96% | | |
| commercial conditioner-1² + 40 | | |
| washes with commercial shampoo-1³ | | |

¹commercial color-1 ingredients: Water, PEG-4 Rapeseedamide, Alcohol Denat, Glyceryl Lauryl Ether, Deceth-3, Propylene Glycol, Laureth-5 Carboxylic Acid, Ethanolamine, Dipropylene Glycol, Hexylene Glycol, Ammonium Hydroxide, Polyquaternium-6, 4-Amino-2-Hydroxytoluene, Oleyl Alcohol, Parfum/Fragrance, p-aminophenol, ammonium thiolactate, p-Phenylenediamine, Erythorbic Acid, EDTA, Polyquaternium-24, 2-Methyl-5-Hydroxy-ethylaminophenol, 6-hydroxyindole, Resorcinol.
²Commercial conditioner-1 ingredients; Cetearyl Alcohol/Behentrimonium Methosulfate, Dimethicone Silylate/Isododecane, Stearalkonium Chloride, Cetearyl Alcohol/Behentrimonium Chloride, Cetyl Alcohol, Helianthus Annuus (Suflower) Seed Oil, Tricaprylyl Citrate, Purified Water, Polyquaternium-10, Hydroxypropyl Starch Phosphate, Glycerin, Sodium Gluconate, Ethyl Macadamiate, Helianthus Annuus (Suflower) Seed Extract, Water (Aqua Purifita) Purified/Foeniculum Vulgare (Fennel) Seed Extract, Water/Aqua/Eau/Wheat Amino Acids/Hydrolized Brazil Nut Protein, Water/Aqua/Eau/Wheat Amino Acids/Sodium Chloride/Hydroxypropyltrimonium Hydrolyzed Wheat Protein/Hydrolized Brazil Nut Protein, Tocopherol, Phenoxyethanol, Aspalathus Linearis Leaf Extract/Maltodextrin, Simmondsia Chinensis (Jojoba) Seed Oil.
³Commercial shampoo-1 ingredients; Water, Sodium Lauryl Sulfate, Sodium Laureth Sulfate, Sodium Chloride, Glycol Distearate, Cocamidopropyl Betane, Dimetjicone, Citric acid, Cocamide MEA, Sodium citrate, Fragrance, Sodium Xylenesulfonate, Sodium Benzoate, Guar Hydroxypropyltrimonium chloride, Tetrasodium EDTA, Panthenol, Panthenyl Ethyl Ether, Methylchloroisothiazolinone, Methyllisothiazolinone.

The results shown in Table 1 demonstrate that natural (non-color-treated hair) is hydrophobic (water contact angle of 103.7±2.2 degrees), while hair treated with a conventional hair dye product is hydrophilic (water contact angle of 65±1.2 degrees), indicating that the coloring process damages the hair cuticle. Treatment of colored hair with a conventional hair conditioner partially restores the hair's hydrophobicity. The contact angle results show that treatment of colored hair with a composition according to the present invention comprising color-lock conditioning technology repairs the color-treated hair surface, restoring the hair's natural hydrophobic characteristics (water contact angle of 127±2.1 degrees), and further that, even after 40 washes (shampoo/comb/blow drying cycles) of the hair treated with a composition according to the present invention, the hydrophobicity is maintained.

### Example 3

### Persistence of Thiolated Polymer Film on the Hair

Gravimetric Analysis was employed to measure the persistence of the polymer film on the hair colored swatches as a function of washing with shampoo, combing, and blow drying. The inventors hypothesized that a thiolated polymer system having the following characteristics would demonstrate superior persistence on the hair (wash-resistance):
Maximum binding to hair to withstand multiple washes shampoo/combing/blow drying cycles (up to 40+).
95-100% of residual polymer film remaining on the surface of the colored hair after multiple washes (shampoos/combing/blow drying) cycles.
Locking-in hair color, i.e., hair color remaining the same, even after multiple (up to 40+) shampoo/combing/blow drying cycles.

Procedure:

1. Eleven swatches of 2-3 g colored hair (colored in accordance with the procedure in Example 2 herein) were weighed out using a Sartorious 1615 MP Micro-balance.

2. Test Materials (see Table 2, below) were applied on the surface of each colored hair swatch and the hair was blown dry.

3. The weight of the Test Material on the surface of each colored hair swatch was determined using the Sartorious 1615 MP Micro-balance: Wt._{Test Material}=Wt._{Test Material+hair)}−Wt.ₕₐᵢᵣ.

4. Each Test Material-treated colored hair swatch was washed with commercial shampoo-1, and dried with a blow dryer. After each shampoo/combing/blow dry cycle, the hair was reweighed.

5. After 40 shampoo/combing/blow dry cycles, the final weight of the Test Material treated colored hair was determined: Wt._{Test Material remaining after 40 washes}=(Wt._{initial Test Material treated colored hair}−Wt._{Test Material treated colored hair after 40 washes}).

Results are shown in Table 3, below.

TABLE 2

| Test | |
|---|---|
| Mate- | |
| rial | Test Material Ingredients |
| 1 | 50 wt. % Dimethicone/mercapto propyl/methiconol copolymer (1- |
| | 3% reactive thiol pendant groups) |
| | 20 wt. % DC 7-4405 (dimethicone silylate/isododecane film |
| | former) |
| | 10 wt. % Covacryl P-12 (hydrophobically modified acrylate |
| | polymer) |
| | 5 wt. % Fucogel II BPC (polysaccharide) |
| | 5 wt. % Lipidure PMB pH 10 (Polyquaternium-51) |
| | 10 wt. % vinyl caprolactam/EthylAminopropylethylamine |
| | copolymer) |
| 2 | 50 wt. % Dimethicone/mercapto propyl Methicone copolymer |
| | (11% reactive thiol pendant groups) |
| | 20 wt. % DC 7-4405 (dimethicone silylate/isododecane film |
| | former) |
| | 1 wt. 0% Covacryl P-12 (hydrophobically modified acrylate |
| | polymer) |
| | 5 wt. % Fucogel II BPC (polysaccharide) |
| | 5 wt. % Lipidure PMB pH 10 (Polyquaternium-51) |
| | 10 wt. % vinyl caprolactam/EthylAminopropylethylamine |
| | copolymer |
| 3 | 50 wt. % Dimethicone/Mercapto propyl Methicone Copolymer |
| | (and) Phenyl Trimethicone (11% reactive thiol pendant groups) |
| | 20 wt. % DC 7-4405 (dimethicone silylate/isododecane film |
| | former) |
| | 10 wt. % Covacryl P-12 (hydrophobically modified acrylate |
| | polymer) |
| | 5 wt. % Fucogel II BPC (polysaccharide) |
| | 5 wt. % Lipidure PMB pH 10 (Polyquaternium-51) |
| | 1 wt. 0% vvinyl caprolactam/EthylAminopropylethylamine |
| | copolymer |
| 4 | 50 wt. % Dimethicone/mercapto propyl Methicone copolymer |
| | (11% reactive thiol pendant groups) |
| | 20 wt. % DC 7-4405 (dimethicone silylate/isododecane film |
| | former) |
| | 10 wt. % Covacryl P-12 (hydrophobically modified acrylate |
| | polymer) |
| | 2 wt. % Fucogel II BPC (polysaccharide) |
| | 2.5 wt. % Aquaflex XL-30 (polyimide-1) |
| | 0.5 wt. % Transglutaminase |
| | 5 wt. % Lipidure PMB pH 10 (Polyquaternium-51) |
| | 10 wt. % vinyl caprolactam/EthylAminopropylethylamine |
| | copolymer) |
| 5 | 40 wt. % Dimethicone/Mercapto propyl Methicone Copolymer |
| | (and) Phenyl Trimethicone (11% reactive thiol pendant groups) |
| | 20 wt. % DC 7-4405 (dimethicone silylate/isododecane film |
| | former) |
| | 19.5 wt. % Aquaflex XL-30 (polyimide-1) |
| | 0.5 wt. % Transglutaminase |
| | 15 wt. % L-Cysteine |
| | 5 wt. % vinyl caprolactam/EthylAminopropylethylamine |
| | copolymer |
| 6 | 40 wt. % Dimethicone/Mercapto propyl Methicone Copolymer |
| | (and) Phenyl Trimethicone (11% reactive thiol pendant groups) |
| | 20 wt. % DC 7-440 5(dimethicone silylate/isododecane film |
| | former) |
| | 19.5 wt. % Aquastyle 3000 (Polyvinylamide-1) |
| | 0.5 wt. % Transglutaminase |
| | 15 wt. % L-Cysteine |
| | 5 wt. % vinyl caprolactam/ethylaminopropylethylamine |
| | copolymer) |
| 7 | 30 wt. % Dimethicone/Mercapto propyl Methicone Copolymer |
| | (and) Phenyl Trimethicone (11% reactive thiol pendant groups) |
| | 20 wt. % DC 7-440 5(dimethicone silylate/isododecane film |
| | former) |
| | 19.5 wt. % Aquaflex XL-30 (polyimide-1) |
| | 0.5 wt. % Transglutaminase |
| | 25 wt. % L-Cysteine |
| | 5 wt. % vinyl caprolactam/EthylAminopropylethylamine |
| | Copolymer |
| 8 | 30 wt. % Dimethicone/Mercapto propyl Methicone Copolymer |
| | (and) Phenyl Trimethicone (11% reactive thiol pendant groups) |
| | 20 wt. % DC 7-4405 (dimethicone silylate/isododecane film |
| | former) |
| | 19.5 wt. % Aquastyle 3000 (Polyvinylamide-1) |
| | 0.5 wt. % Transglutaminase |
| | 25 wt. % L-Cysteine |
| | 5 wt. % vinyl caprolactam/EthylAminopropylethylamine |
| | copolymer) |
| 9 | 25 wt. % Dimethicone/mercapto propyl Methicone copolymer |
| | (11% reactive thiol pendant groups) |
| | 25 wt. % Phenyl Mercapto (11% Pendant SH) |
| | 50 wt. % DC7-4405 (dimethicone silylate/isododecane film |
| | former) |
| 10 | 4 wt. % Test Material 9 |
| | 2 wt. % Styleze W-17 (Polyquaternium-55) VP/dimethyl- |
| | aminopropyl methacrylamide/ |
| | methacryloylaminopropyllauryldimonium chloride copolymer |
| | 1 wt. % Pantethine/Pantethine A |
| | 0.5 wt. % Ethyl Panthenyl Ether |
| | 92.5 wt. % commercial conditioner-2 |
| 11 | 4 wt. % Test Material 9 |
| | 96 wt. % commercial conditioner-2 |

¹ commercial conditioner-2 ingredients: Cetearyl Alcohol/Behentrimonium Methosulfate, Dimethicone Silylate/Isododecane, Stearalkonium Chloride, Cetearyl Alcohol/Behentrimonium Chloride, Cetyl Alcohol, Helianthus Annuus (Sunflower) Seed Oil, Tricaprylyl Citrate, Purified Water, Polyquaternium-10, Hydroxypropyl Starch Phosphate, Glycerin, Sodium Gluconate, Ethyl Macadamiate, Helianthus Annuus (Sunflower) Seed Extract, Water (Aqua Purifita) Purified/Foeniculum Vulgare (Fennel) Seed Extract, Water/Aqua/Eau/Wheat Amino Acids/Hydrolyzed Brazil Nut Protein, Water/Aqua/Eau/Wheat Amino Acids/Sodium Chloride/Hydroxypropyltrimonium Hydrolyzed Wheat Protein/Hydrolyzed Brazil Nut Protein, Tocopherol, Phenoxyethanol, Aspalathus Linearis Leaf Extract/Maltodextrin, Simmondsia Chinensis (Jojoba) Seed Oil.

TABLE 3

| | Percent Test Material Remaining After | | | | | | | | |
|---|---|---|---|---|---|---|---|---|---|
| | Shampoo¹/Comb/Blow Dry (S/C/BD) Cycle(s) | | | | | | | | |
| | % | % | % | % | % | % | % | % | % |
| | after | after | after | after | after | after | after | after | after |
| Test | S/C/ | S/C/ | S/C/ | S/C/ | S/C/ | S/C/ | S/C/ | S/C/ | S/C/ |
| Material | BD1 | BD2 | BD3 | BD4 | BD5 | BD10 | BD15 | BD30 | BD40 |
| 1 | 84 | 84 | 66 | 66 | 66 | 66 | 66 | 66 | 52 |
| 2 | 100 | 86 | 86 | 86 | 86 | 86 | 86 | 86 | 86 |
| 3 | 92 | 87 | 87 | 87 | 87 | 87 | 87 | 87 | 87 |
| 4 | 92 | 88 | 69 | 67 | 66 | 60 | ND² | ND | ND |
| 5 | 95 | 95 | 64 | 64 | 64 | 64 | ND | ND | ND |
| 6 | 78 | 77 | 77 | 77 | 77 | 77 | ND | ND | ND |
| 7 | 100 | 89 | 83 | 83 | 83 | 55 | ND | ND | ND |
| 8 | 95 | 86 | 80 | 75 | 70 | 70 | ND | ND | ND |
| 9 | 100 | 100 | 100 | 100 | 100 | 100 | 100 | 100 | 100 |
| 10 | 100 | 100 | 100 | 100 | 100 | 100 | 100 | 100 | 100 |
| 11 | 100 | 100 | 100 | 100 | 100 | 100 | 100 | 100 | 100 |

¹commercial shampoo-1
²not done

Table 3 demonstrates the persistence (wash-resistance) of the Test Materials associated with the hair cuticle after multiple shampoo/comb/blow dry cycles. Surprisingly, up to 100% of Test Materials remain associated with the hair cuticle even after up to 40 shampoo/coming/blow dry cycles. The strongest wash-resistance (100%) after 40 shampoo/comb/blow dry cycles was demonstrated by Test Materials 9-11. The inventors theorize that the ability of the Test Materials to form and maintain a hydrophobic film on the hair cuticle and thus retain the integrity of the film is dependent on the compatibility of the polymers (i.e., the cohesive forces among the polymers, such as through hydrogen bonding) in the test material. It is believed that if the cohesive forces are weak, such as when a Test Material contains a blend of polymers having different degrees of hydrophobicity/hydrophilicity, the polymers may separate on the hair surface and be more easily washed away. On the other hand, when the cohesive forces are strong (i.e., the polymers are compatible), and the Test Material demonstrates an overall greater hydrophobic than hydrophilic character, the integrity of the film on the hair surface will be maintained longer and the adhesion of the film on the hair surface will remain stronger wash after wash. Test Materials 9-11, which performed particularly well, include blends of silicone polymers which are all compatible and hydrophobic. It is to be noted that Test Material 1, which contains a thiolated polymer having no more than 3% reactive thiol groups did not perform as well as Test Materials containing a thiolated polymer having a greater percentage (e.g., 11%) reactive thiol groups. It would be expected that thiolated polymers, other than thiolated silicones, for example but not limited to, at least one thiolated hydrophobically modified acrylates polymer, optionally in combination with at least one other compatible hydrophobic or hydrophobically modified polymer, should also provide a film on the hair which demonstrates superior wash resistance.

### Example 4

### Scanning Electronmicroscopy (SEM) Analysis

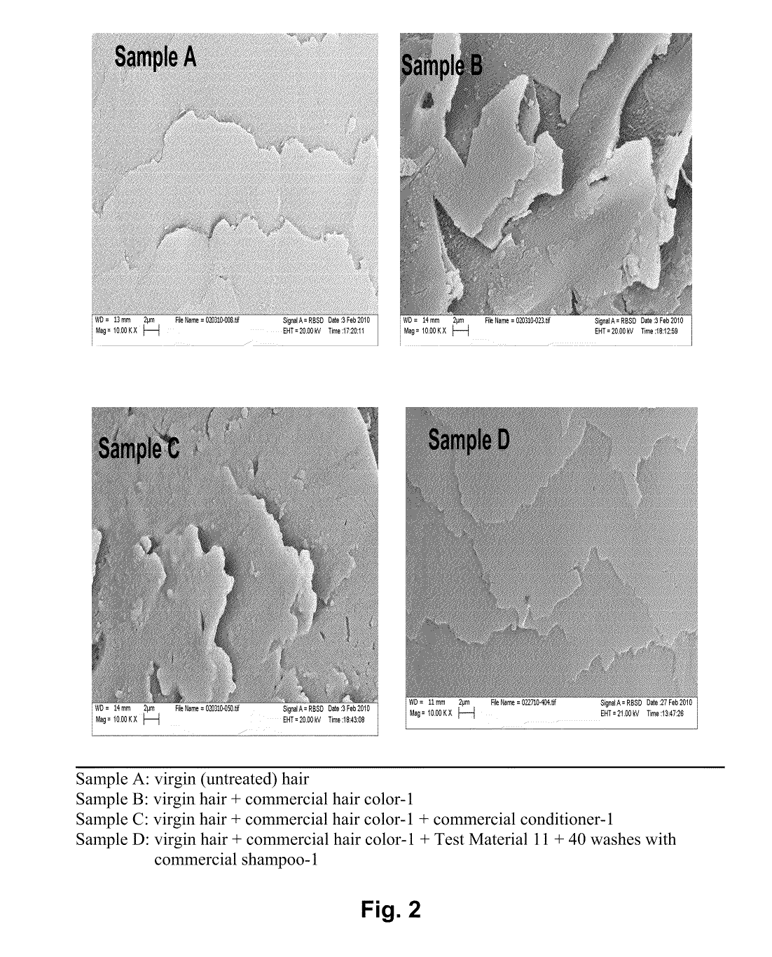

SEM analysis of the surface morphology/topography of the hair fiber surface was used to observe the uniformity, integrity, and adhesion properties of a film formed by the thiolated polymer-containing compositions of the present invention on the hair fiber surface. As shown in FIG. 2, Sample A is an SEM image of (virgin grey) noncolored hair showing a smooth surface. Sample B is an SEM image of the damaged surface of hair colored with commercial hair color-1. Sample C is an SEM image of the damaged surface of hair colored with commercial hair color-1 and then treated with commercial conditioner-1. Sample D is an SEM image of the surface of hair colored with commercial hair color-1, then treated with Test Material 11 and subsequently subjected to 40 cycles of washing (with commercial shampoo-1), combing and blow drying. The SEM images in FIG. 2 show that a composition according to the present invention provides a film having a strong adhesion to the hair surface (Sample D), demonstrating that after 40 cycles of shampooing/combing/blow drying, hair treated with a composition according to the present invention repairs the damaged color treated hair cuticle (i.e., the hair cuticles of Sample D are as smooth as the cuticles of untreated hair in Sample A).

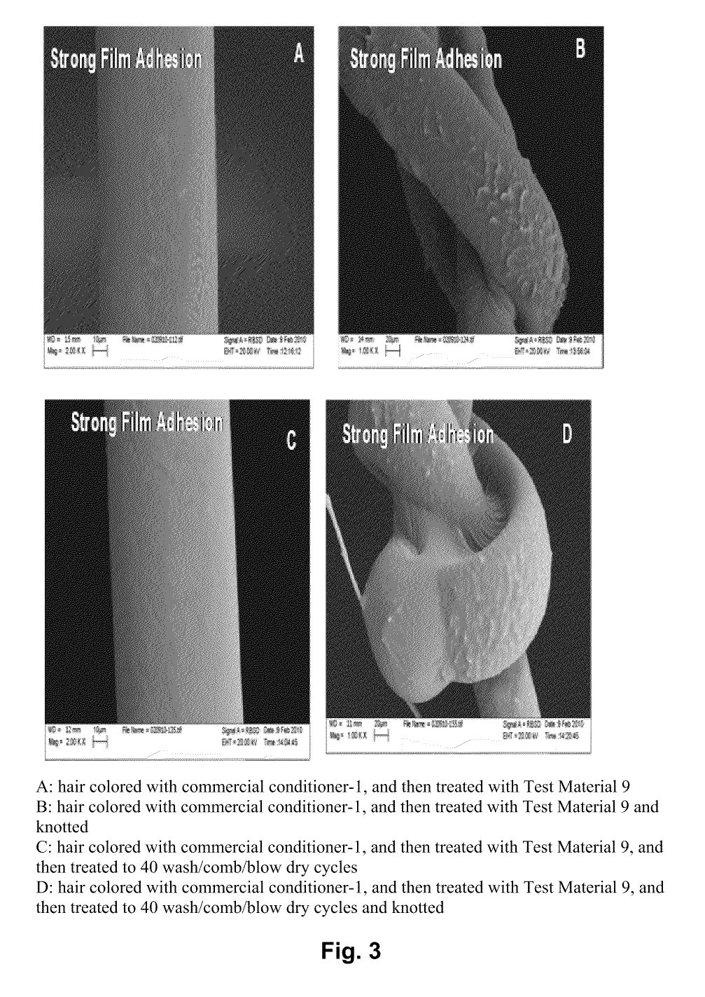

Referring to the SEM images in FIG. 3, Sample A is an image of a single virgin grey which has been colored with commercial hair color-1 and treated with Test Material 9. Sample B is an image of the hair in Sample A in which the hair has been twisted into a knot. Sample C is an image of a single virgin grey hair which has been colored with commercial hair color-1, treated with Test Material 9 and subjected to 40 wash/combing/blow dry cycles. Sample D is an image of the hair in Sample C in which the hair has been twisted into a knot. The images show that a polymer film according to the present invention demonstrates strong adhesion to the hair surface, before washing with shampoo (Samples A and B) as well as after 40 shampoo/combing/blow dry cycles (Samples C and D).

The SEM Images show that the surface of the hair which has been color-treated and then treated with a composition according to the present invention is as smooth as the untreated hair surface, before and after 40 wash/comb/blow dry cycles. Visual observation of the colored hair treated in accordance with the present invention shows that the smoothness of the hair cuticles is associated with improved hair shine.

### Example 5

### Hair Color Retention Analysis

The color retention (DE) efficacy of the color-lock technology of the present invention was measured according to the following procedure:

### A. Hair Color Application

A 15-20 g swatch of virgin grey hair was colored using commercial hair color-1 following package instructions.
The hair swatch was washed in the sink with tap running water until the water became completely clear.

### B. Test Material Application

2.5-3 g (20-25 cm length and 1 cm width) of the hair color swatch, taken from A, above. was damp dried with a paper towel.
1-1.2 g of Test Material was applied thoroughly by hand through the hair swatch and allowed to set (form a film) for 1-2 minutes.
The swatch was then washed with running tap water until the water became completely clear, damp dried with a paper towel, and then combed and blown dry for 10 minutes.

### C: DE Measurement Procedure

A GretagMacneth Spectrolino 8 mm Spectrophotometer Instrument with Colorimeter software Optiview ProPalette 5.2.10. was used for the hair color retention (DE) measurements. Color readings of Hunter L, a, b parameters were obtained with D65 light source setting on Leneta Black Paper Template. A 1 cm² template was used for all the DE measurements.

L, a, b color space is defined as a color opponent space with dimension L for lightness a and b for the color-opponent dimensions, based on nonlinearly-compressed color space coordinates. The three coordinates represent: the lightness of the color, L (L=0 yields black and L=100 indicates diffuse white; specular white may be higher), its position between red and green, a value (negative a values indicate green while positive a values indicate red) and its position between yellow and blue, b value (negative b values indicate blue while positive b values indicate yellow. Color difference was measured according to the following equation: DE=((Lₓ-L_{y})²+(aₓ-a_{y})²+(bₓ-b_{y})²)^{1/2}; where x means after 40 shampoos, and where y means before 40 shampoos. A lower DE value is indicative of greater color retention.

For the Control DE Measurement:

1. A 2.5-3 g swatch of dried hair, colored with commercial hair color-1, was wetted with running tap water.

2. Five L, a, b measurements were obtained across one side of the control swatch, and five L, a, b measurements were obtained the other side of the control swatch. Thus, ten DE values were recorded and the average DE value was reported with error bars. It was observed that all the L, a, b values were very similar for the control swatch (as the hair color was uniformly formed).

For the Pre-Wash Test Materials DE Measurements:
1. A 2.5-3 g swatch of dried hair, colored with commercial hair color-1 according to package directions, was wetted with running tap water.
2. 1-1.2 g of Test Material was applied thoroughly by hand through the hair swatch and allowed to set (form a film) for 1-2 minutes.
3. The Test Material-treated colored hair swatch was washed in the sink with running tap water until the water became completely clear, then damp dried with a paper towel, and then combed and blown dry for 10 minutes.
4. Five L, a, b measurements were obtained across one side of the Test Material-treated hair swatch, and 5 L, a, b measurements were obtained across the other side of the Test Material-treated colored hair swatch. Thus, 10 DE values were recorded and the average DE value was reported with error bars.

For the Post (40 Wash) Test Materials DE Measurements:

1. The Test Material-treated colored hair swatch was washed with commercial shampoo-1 and rinsed well with running tap water until the water became completely clear, then damp dried with a paper towel, and combed and blown dry for 10 minutes.

2. Five L, a, b measurements were obtained across one side of the swatch, and 5 L, a, b measurements were obtained across the other side of the swatch. Thus, ten DE values were recorded, and the average DE value was reported with error bars.

The hair color retention (DE) results are shown in Table 4, below.

TABLE 4

| | Hair Color Retention (DE) |
|---|---|
| Hair Treatment | after 40 washings |
| Commercial Color-1 | 13.762 ± 3.56 |
| Commercial Color-1 + | 3.585 ± 0.11 |
| Commercial Conditioner-1 | |
| Commercial Color-1 + | 4.793 ± 0.32 |
| Commercial Conditioner-2 | |
| Commercial Color-1 + | 4.535 ± 1.27 |
| Commercial Conditioner-3¹ | |
| Commercial Color-1 + Test | 0.989 ± 0.44 |
| Material 10 | |
| Commercial Color-1 + Test | 2.014 ± 0.85 |
| Material 11 | |

¹Commercial Conditioner-3: Water, Stearyl alcohol, Cyclopentasiloxane, Cetyl alcohol, Stearamidopropyl dimethylamine, Imidazolidinyl Urea, Dimethicone, Cyclohexasiloxane, Aspartic acid, DMDM Hydantoin, Quaternium 18, Fragrance (Parfum), Citric acid, Isostearamidopropyl Ethyldimonium Ethydimonium Ethosulfate, Butylphenyl methyl propionol, Limonene, Disodium EDTA, Amyl Cinnamal, PEG-9, Linalool, Geraniol, Hexyl Cinnamal, Camellia Sinensis Leaf Extract, Helianthus Annuus (sunflower) Seed oil, Glyceryn, Polysorbate 20, Hydrolized keratin, Rosmarinus Officinalis (Rosemary) Leaf Extract, Tocopheryl Acetate, Panthenol, Ascorbic Acid, Niacinamide, Biotin.

As shown in Table 4, using the color-lock technology of the present invention, the hair color retention (DE) value was improved from 13.762±3.523 (hair colored using commercial hair color-1) to 0.989±0.44 (hair colored using commercial hair color-1 and then treated with Test Material 10), and to 2.014±0.85 (hair colored using commercial hair color-1 and then treated with Test Material 11). Additionally, after 40 washes with commercial shampoo-1, the hair treated to the Test Materials containing the color-lock technology of the present invention retained color to a substantially greater degree (lower DE values) than does hair treated with any of commercial conditioners 1, 2 and 3. This also indicates that by using the color-lock technology, the hair color stays true; i.e., does not fade even after 40 washes with commercial shampoo.

### Example 6

### X-Ray Photonelectron Spectroscopy (XPS) Analysis

XPS was used to assess the uniformity of the compositions of the present invention deposited on the hair. XPS is a quantitative spectroscopic technique that measures the elemental composition, empirical formula, chemical state and electronic of the elements that exist within a material. In this assay, XPS was used to measure the binding energy values of electrons detected from the surface of the hair fibers. XPS quantitatively detects the uniformity of the elemental composition of the hair surfaces of the samples (C, H, O, N, S, Si, etc.), as well as the uniformity of color-lock technology films on the colored hair surface.

### Experimental Procedure:

1. XPS (X-ray Photoelectron Spectroscopy) measurements were carried out in an Ultra Axis™ spectrometer, (manufacturer: Kratos Analytical, Manchester UK).
2. The samples were irradiated with monoenergetic Al K_{α1,2} radiation (1486.6 eV) and the spectra were taken at a power of 144 W (12 kV×12 mA).
3. The aliphatic carbon (C—C, C—H) at a binding energy of 285 eV (C 1s photoline) was used to determine the charging.
4. The spectral resolution—i.e. the Full Width of Half Maximum (FWHM) of the Ester carbon from PET—was better than 0.68 eV for the elemental spectra.
5. The elemental concentration is given in atom %; however, this method does not detect hydrogen and helium.

TABLE 5
Elemental Composition (Atomic %) of Samples 1-5

| | Sample | | | | |
|---|---|---|---|---|---|
| Elements | 1 | 2 | 3 | 4 | 5 |
| C | 66.8 | 71.0 | 68.5 | 55.6 | 59.2 |
| O | 21.2 | 17.0 | 18.4 | 27.2 | 22.7 |
| N | 4.4 | 7.3 | 6.0 | — | 4.1 |
| S | 0.4 | 2.4 | 1.2 | — | 1.4 |
| Si | 2.8 | 1.9 | 5.6 | 16.8 | 12.6 |
| Other | 4.4 | 0.4 | 0.3 | 0.4 | 0 |

Sample 1: Virgin Grey Hair
Sample 2: Virgin Grey Hair + Commercial Color-1
Sample 3: Virgin Grey Hair + Commercial Color-1 + Commercial Conditioner-2
Sample 4: Virgin Grey Hair + Commercial Color-1 + Test Material 11
Sample 5: Virgin Grey Hair + Commercial Color-1 + Test Material 11 + 40 Washes with Commercial Shampoo

As shown in Table 5, a significant increase of the Si content over that of virgin grey hair was detected in samples 4 and 5 due to their treatment with the silicone-containing Test Materials 10 or 11. It is notable that most of the silicon polymer remains associated with the hair surface even after 40 washings. This indicates, indirectly, that the silicon polymer is very strongly bonded on the hair surface, suggesting covalent bonding.

### Example 7

### Disulfide Bond Colorimetric Assay

The hair cystine content, as indicated by the presence of disulfide bonds, was quantified by measuring the amount of oxidized dithiothreitol (DTT) (λmax=280 nm) derived from DTT with cystine.

### Procedure:

1. 15-20 mg hair samples were weighed out using a Sartorius 1615 Micro-balance. Hair samples were treated as follows:
   a. Untreated (virgin grey hair).
   b. Colored with commercial hair color-1 according to package directions.
   c. Colored with commercial hair color-1, then treated with commercial conditioner-1.
   d. Colored with commercial hair color-1, then treated with Test Material 10 and then rinsed with running tap water and dried with a blow dryer for 10 minutes.
   e. Colored with commercial hair color-1, then treated with Test Material 11.
2. Each hair sample was weighed out again.
3. Each hair sample was sonicated twice for 15 minutes in a solution of water:ethanol (1:1).
4. Each hair sample was dried in air for 24 hours.
5. A mixture of 1.54 g of dithiothreitol (DTT) in 100 mL of DI water was prepared.
6. Each hair sample was deposited in polypropylene tube containing 1.3 mL of the DTT-water solution and the tube containing the hair/DTT-water mixture was incubated in an oven at 60 degree C. for 16 hours.
7. Each supernatant solution was diluted in water and 200 microliters of the solution dispensed into a UV plate well to measure the absorbance of the DTT at 280 nm wavelength. The absorbance peak at 280 nm wavelength is a direct measurement of the amount oxidized DTT derived from DTT and cystine. (The higher the peak the more cystine (S—S) bonds in the hair).
8. The hair cystine (S—S) content of each sample was calculated using an oxidized glutathione standard curve solution.

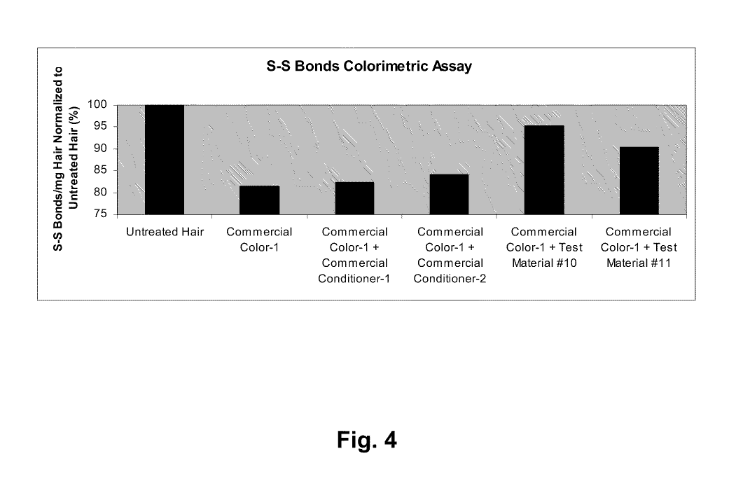

The results shown in FIG. 4 demonstrate that, taking a value of 100% S—S bonds for the untreated hair surface, the color-treated hair surface retained 80% S—S bonds, and color-treated hair which also was treated with a color-lock composition according to the present invention indicated the presence of 95% S—S bonds. This indicates that the color-lock technology (test Material 10) reforms the S—S bonds of the color treated surface, and repairs the damaged surface of color hair.

### Example 8

### Film Adhesion/Thickness Analysis

Atomic Force Microscopy (AFM) and Micro-indentation techniques were used to determine the thickness, adhesion strength, softness/stiffness, as well as surface morphology of the color-lock technology film forming characteristics on colored-hair surface.

### Atomic Force Microscopy Procedure:

1. The samples were prepared by mounting with double-sided adhesive tape to sapphire substrates. Tape was mounted in two locations of the sapphire substrate and hair fibers laid across the two regions.
2. The measurement location was between the two tape regions, and the spacing was found sufficient such that fibers were stable and did not slide or move when loads were applied.
3. The fiber in direct contact with the sapphire substrate provides a stiff contact for the hair fibers. AFM force spectroscopy was performed on the samples, and a sapphire reference material.
4. The AFM probe is brought in to contact with the fiber by landing on the surface of the material (either hair fiber or reference substrate). Measurements were located at the apex of the fiber.
5. The cantilever displacement is increased to induce deformation of the cantilever beam and create force at the probe tip and material contact.
6. The displacement was increased to increase the applied load and penetrate the material.
7. The slope of the loading curve is analyzed for the apparent stiffness of the surface.

Micro-Indentation Procedure:
1. Measurement of coating thickness on the surface of the hair fibers was performed using instrumented-microindentation (MicroMaterials MicroTest) with a Berkovich diamond probe tip.
2. Samples were mounted on polished aluminum sample stub surfaces using double sided adhesive, as described in the materials section. Calibration of the load, displacement, and microscope position were made prior to indentation testing.
3. The instrument was calibrated using a polished fused quartz sample to verify proper operation. Sample stubs with mounted fibers were placed in the instrument and individual fibers were located with the in situ optical microscope.
4. Instrumented-indentation was performed on the apex of the hair fiber to create a consistent contact area. The center of the cuticle between two edges was the spatial location of the measurement along the fiber axis.
5. Depth-controlled indentations were performed to a range of depths from 5 to 15 μm.
6. The instrument records simultaneous real-time measurement of tip displacement and normal load.
7. Normal load versus tip displacement for each hair fiber sample was determined.

The Results are the Following:

Micro-indentation results (not shown) indicate a color-lock technology film thickness on the order of 8-9 microns on hair colored with commercial hair color-1. A similar film thickness was measured for the commercial conditioner-2. This indicates that the color lock polymer system of the present invention is compatible with the commercial conditioner-2 base, i.e., it forms a continuous and uniform film on colored hair (sealing the ingredients together), and spreads well on hair without building viscosity.

Additionally, the color-lock film on the colored hair is stiffer (i.e., more rigid, less flexible) than that of the commercial conditioner-2. Furthermore, the color-lock film demonstrates higher adhesion strength to the colored hair than that of the commercial conditioner-2. Greater adhesion results in high hair color retention even after 40 cycles of washing/combing/blow drying.

AFM images analysis (not shown) demonstrates that the color-lock film surface morphology is as smooth and uniform as the control virgin hair, and further that color-lock technology is smoother and more uniform than that of the commercial conditioner-2 alone. The colored-hair surface (not further treated) shows the most non-uniform surface.

The results of these assays indicate that color-lock technology repairs the surface of the colored hair. These results are consistent with the results of the scanning electron microscopy analysis.

### Example 9

### Hair Conditioner (Test Material 10)

| | |
|---|---|
| Materials | Weight % |

Phase 1

| | |
|---|---|
| Cetearyl alcohol/behentrimonium methosulfate | 2.010 |
| Dimethicone silylate/isododecane | 2.000 |
| Stearalkonium chloride | 1.150 |
| Cetearyl alcohol/behentrimonium chloride | 3.500 |
| Cetyl alcohol | 2.000 |
| Sunflower seed oil | 0.250 |
| Tricaprylyl citrate | 0.500 |

Phase 2

| | |
|---|---|
| Deionized water | 78.779 |

Phase 3

| | |
|---|---|
| Polyquaternium-10 | 0.300 |
| Hydroxypropyl starch phosphate | 0.750 |

Phase 4

| | |
|---|---|
| Glycerin | 1.000 |
| Sodium gluconate | 0.200 |

Phase 5

| | |
|---|---|
| Ethyl macadiate | 0.500 |
| Sunflower seed extract | 0.010 |
| Fennel seed extract | 0.500 |
| Hydrolyzed brazil nut protein/wheat amino acids/ | 0.050 |
| water | |
| Hydrolyzed brazil nut protein/hydroxypropyl | 0.025 |
| Trimonium hydrolyzed wheat protein/ | |
| wheat amino acids/sodium chloride/water | |

Phase 6

| | |
|---|---|
| Tocopherol (Vitamin E) | 0.010 |
| Phenoxyethanol | 0.500 |
| Aspalathus Linearis leaf extact/maltodextrin | 0.001 |
| Simmondsia chineensis (jojoba) seed oil | 0.120 |
| Pantethine | 1.000 |
| Panthenyl ethyl ether | 0.500 |
| Ricinus communis (castor) seed oil | 0.120 |
| Polyquaternium-55/caprylyl glycol/water | 2.000 |
| Dimethicone/mercaptopropyl methicone copolymer | 1.000 |
| Dimethicone/mercaptopropyl methicone copolymer/ | 1.000 |
| Phenyltrimethicone | |
| TOTAL | 100.000 |

The hair conditioner was prepared as follows:
1. Phase 2 and 3 ingredients were added to a main kettle, and mixed at room temp until clear and uniform.
2. The temperature of the phase 1 mixture was increased to 82-85° C.
3. Phase 1 ingredients were added to a separate kettle, and the temperature was increased to 82-85° C., with mixing until clear.
4. The Phase 1 mixture was added to the mixture in the main kettle, and the temperature gradually reduced while mixing.
5. Mixing was continued, and at 60° C., the phase 4 ingredient was added.
6. Mixing was continued, and at 40° C., the phase 5 ingredients were added, one by one.
7. Mixing was continued while further reducing the temperature to 30° C.
8. At 30° C., the Phase 6 ingredients were added, one by one, and mixed until uniform.
9. When the batch temperature reached 25-30° C., the batch was mixed (Silverson) at 4000 rpm for 5 minutes.

### Example 10

### Hair Conditioner

| | |
|---|---|
| Materials | Weight % |

Phase 1

| | |
|---|---|
| Cetearyl alcohol/behentrimonium methosulfate | 2.010 |
| Dimethicone silylate/isododecane | 2.000 |
| Stearalkonium chloride | 1.150 |
| Cetearyl alcohol/behentrimonium chloride | 3.500 |
| Cetyl alcohol | 2.000 |
| Sunflower seed oil | 0.250 |
| Tricaprylyl citrate | 0.500 |

Phase 2

| | |
|---|---|
| Deionized water | 76.779 |

Phase 3

| | |
|---|---|
| Polyquaternium-10 | 0.300 |
| Hydroxypropyl starch phosphate | 0.750 |

Phase 4

| | |
|---|---|
| Glycerin | 1.000 |
| Sodium gluconate | 0.200 |

Phase 5

| | |
|---|---|
| Ethyl macadiate | 0.500 |
| Sunflower seed extract | 0.010 |
| Fennel seed extract | 0.500 |
| Hydrolyzed brazil nut protein/wheat amino acids/ | 0.050 |
| water | |
| Hydrolyzed brazil nut protein/hydroxypropyl | 0.025 |
| Trimonium hydrolyzed wheat protein/ | |
| wheat amino acids/sodium chloride/water | |

Phase 6

| | |
|---|---|
| Tocopherol (Vitamin E) | 0.010 |
| Phenoxyethanol | 0.500 |
| Aspalathus Linearis leaf extact/maltodextrin | 0.001 |
| Simmondsia chineensis (jojoba) seed oil | 0.120 |
| Pantethine | 1.000 |
| Panthenyl ethyl ether | 0.500 |
| Ricinus communis (castor) seed oil | 0.120 |
| Polyquaternium-55/caprylyl glycol/water | 2.000 |
| Dimethicone/mercaptopropyl methicone copolymer | 2.000 |
| Dimethicone/mercaptopropyl methicone copolymer/ | 2.000 |
| Phenyltrimethicone | |
| TOTAL | 100.000 |

The hair conditioner was prepared as in Example 9.

It should be understood that the foregoing relates to certain preferred embodiments of the present invention and that numerous modifications or alterations maybe made therein without departing from the spirit and scope of the invention.

What is claimed is:

1. A cosmetic hair treatment composition comprising at least one thiolated polymer having from greater than about 3% to about 50% reactive thiol groups, or a polymer blend comprising the at least one thiolated polymer, in a cosmetically or dermatologically acceptable vehicle.

2. The cosmetic hair treatment composition of claim 1 wherein the at least one thiolated polymer has in the range of from about 5% to about 50% reactive thiol groups.

3. The cosmetic hair treatment composition of claim 1 wherein the at least one thiolated polymer has in the range of from about 10% to about 30% reactive thiol groups.

4. The cosmetic hair treatment composition of claim 1 wherein the at least one thiolated polymer or polymer blend comprising the at least one thiolated polymer is present in the composition in an amount in the range of from about 2 wt. % to about 50 wt. % by weight of the total composition.

5. The cosmetic hair treatment composition of claim 1 wherein the at least one thiolated polymer is present in the polymer blend in an amount in the range of from about 30 wt. % to about 80 wt. % by total weight of the polymer blend.

6. The cosmetic hair treatment composition of claim 5 wherein the at least one thiolated polymer is present in the polymer blend in an amount in the range of from about 30 wt. % to about 50 wt. % by total weight of the polymer blend.

7. The cosmetic hair treatment composition of claim 1, wherein the thiolated polymer is a homopolymer which is selected from the group consisting of polystyrene, polyester, polyfluoroester, polyethylene, polypropylene, polybutadiene, polyisoprene, polyurethane, polyimide, silicones, hydrophobically modified polyacrylates, hydrophobically modified hyaluronic acid, hydrophobically modified cellulose, hydrophobically modified starch, hydrophobically modified polysaccharides, hydrophobically modified polyvinyl pyrrolidone, hydrophobically polyvinyl alcohol, and mixtures thereof.

8. The cosmetic hair treatment composition of claim 7, wherein the at least one thiolated polymer is a mercapto silicone polymer.

9. The cosmetic hair treatment composition of claim 8, wherein the mercapto silicone polymer comprises Dimethicone/mercapto propyl Methicone copolymer, Dimethicone/Mercapto propyl Methicone Copolymer (and) Phenyl Trimethicone, or a combination thereof.

10. The cosmetic hair treatment composition of claim 1, wherein the at least one thiolated polymer is an amphiphilic block copolymer having hydrophobic blocks and hydrophilic blocks, wherein the hydrophobic blocks are selected from the group consisting of polystyrene, polyester, polyfluoroester, polyethylene, polypropylene, polybutadiene, polyisoprene, polyurethane, polyimide, silicones, hydrophobically modified polyacrylates, hydrophobically modified hyaluronic acid, hydrophobically modified cellulose, hydrophobically modified starch, hydrophobically modified polysaccharides, hydrophobically modified polyvinyl pyrrolidone, hydrophobically polyvinyl alcohol, and mixtures thereof, and wherein the hydrophilic blocks are selected from the group consisting of polyvinyl pyrrolidone, hyaluronic acid, polyacrylates, polyvinyl chloride, polysaccharides, cellulose, and mixtures thereof.

11. The cosmetic hair treatment composition of claim 1, wherein the polymer blend comprises at least one film forming polymer selected from the group consisting of silicones, hydrophobically modified acrylates polymers, polysaccharides, polyimides, polyvinyl imides, and combinations thereof.

12. The cosmetic hair treatment composition of claim 1, wherein the polymer blend comprises Dimethicone/mercapto propyl Methicone copolymer, Dimethicone/Mercapto propyl Methicone Copolymer (and) Phenyl Trimethicone, and Dimethicone silylate/isododecane.

13. The cosmetic hair treatment composition of claim 1, comprising at least one further ingredient selected from the group consisting of cosmetic actives, moisturizers, humectants, conditioning agents, antistatic agents, styling/fixative aids, oils, emulsifiers and thickening agents.

14. The cosmetic hair treatment composition of claim 1, which is in the form of an oil/water emulsion, a silicon/water emulsion, a water/oil emulsion, a water/silicone emulsion, or an aqueous preparation.

15. The cosmetic hair treatment of composition of claim 1 which is in the form of a solution, a serum, a gel, a lotion, a mousse, or a cream.

16. A method for extending the color retention of color-treated hair, which comprises applying to the hair in need of the extended color retention a composition comprising at least one thiolated polymer having from about 5% to about 50% reactive thiol groups, or a polymer blend comprising the at least one thiolated polymer, in a cosmetically or dermatologically acceptable vehicle, and retaining the composition on the hair for a time period sufficient to seal color in the color-treated hair.

17. A method according to claim 16, wherein the at least one thiolated polymer has in the range of from about 10% to about 30% reactive thiol groups.

18. A method according to claim 16, wherein the at least one thiolated polymer or polymer blend comprising the at least one thiolated polymer is present in the composition in an amount in the range of from about 5 wt. % to about 50 wt. % by weight of the total composition.

19. A method according to claim 16, wherein the at least one thiolated polymer is a homopolymer comprising Dimethicone/mercapto propyl Methicone copolymer, Dimethicone/Mercapto propyl Methicone Copolymer (and) Phenyl Trimethicone, or a combination thereof.

20. A method for treating the hair to restore one or more of hydrophobicity, smoothness, manageability and brilliance, which comprises applying to the hair in need of such treatment a cosmetic composition comprising at least one thiolated polymer having from about 5% to about 50% reactive thiol groups, or a polymer blend comprising the at least one thiolated polymer, in a cosmetically or dermatologically acceptable vehicle, and retaining the composition on the hair for a time period sufficient to obtain the desired effect.
